(19) United States
(12) Patent Application Publication
(10) Pub. No.: US 2003/0138532 A1
(43) Pub. Date: Jul. 24, 2003

(54) Food and beverage preservative

(76) Inventor: Paul L. Simmons, Pinellas Park, FL (US)

Correspondence Address: Arthur W. Fisher, III, Suite 316, 5553 West Waters Avenue, Tampa, FL, 33634, US

(21) Appl. No.: 10/132,019

(22) Filed: Apr. 24, 2002

Related U.S. Application Data
(60) Provisional application No. 60/350,792, filed on Jan. 22, 2002.

(51) Int. Cl. A61K 31/717; A23L 3/34

(52) U.S. Cl. 426/321; 514/57

(57) ABSTRACT

A food and beverage preservative formulated to reduce growth of microbial contaminants such as bacteria and spores that cause food and beverage spoilage including a vegetable cellulose fiber and an emulsifier having a pH of at least about 7.0 and a method of manufacturing the food and beverage preservative.

### CROSS REFERENCE APPLICATIONS

This is a non-provisional patent application of provisional patent application Serial No. 60/350,792 filed Jan. 22, 2002.

### BACKGROUND OF THE INVENTION

1. Field of the Invention

A food and beverage preservative formulated to reduce growth of microbial contaminants such as bacteria and spores that cause food and beverage spoilage.

2. Description of the Prior Art

Bacteria and other microbial organisms cause food and beverage as well as other products to spoil thereby reducing the shelf life or useful life of such products or goods. Thus, numerous efforts have been made to reduce the deleterious effects of microbial contaminants in organic products and materials.

For example, U.S. Pat. No. 6,103,294 shows a process for producing cellulose pulp and a filtrate of cellulose pulp useful as a preservative for digestible food products. In the emulsification process wax paper, water, preservative potassium sorbate and the surfactant hydroxylated lecithin are heated and blended. The cellulose pulp can be used in various with food products as a preservative or used to rinse fresh fruits and vegetables to increase the time that the fruits or vegetables can be safely stored without refrigeration.

U.S. Pat. No. 5,840,294 and U.S. Pat. No. 5,412,090 disclose a similar method for extending the self life of products such as toothpaste, shampoo, soap, detergent and lotions or creams by adding a hydrous cellulose pulp. The hydrous cellulose pulp is resistant to decomposition and can be produced either with recycled waxed paper or through a process that begins with virgin vegetable constituents and wax.

U.S. Pat. No. 5,840,294 also discloses a cellulose pulp based food preservative including wax paper.

U.S. Pat. No. 6,171,550 B1 shows a similar method for extending the shelf life of products such as toothpaste, shampoo, soap, detergent and lotions or creams by utilizing a water base of about 99% water, 0.68% cellulose and 0.32% emulsified food grade wax. Products produced with this water base will not decompose without chemical preservatives. The hydrous cellulose pulp can be produced either with recycled waxed paper or through a process that begins with virgin vegetable constituents and wax. The filtrate or water base can also be used in the horticultural field to prevent mold on plants and in physiology area to prevent the degradation of cells.

Unfortunately, these earlier examples include non-food grade ingredients such as wax paper used in a cellulose fiber. Moreover, the wax paper generally contains toxic bleaches and whiteners maintained at a pH level of 6.5 or less to be effective against spoilage.

### SUMMARY OF THE INVENTION

The present invention relates to a viscous liquid preservative formulated to reduce growth of microbial contaminants such as bacteria and spores that cause food spoilage. Previous products using similar technology include non-food grade ingredients such as waxed paper as the source of cellulose fiber. Unfortunately, the waxed paper generally contains bleaches and whiteners that are toxic by LD 50 test for toxicity and must be maintained at a pH level of 6.5 or less to prevent the paper from spoiling.

The viscous liquid preservative of the present invention uses vegetable cellulose fiber, beeswax and an emulsifier such as soy lecithin. The preservative is stable at neutral of at least about pH 7.0 and preferably from about pH 7.0 to about pH 7.8.

Since the product contains no toxic chemicals, there are no regulatory limits as to what percentage can be used in a food or beverage. Furthermore, without the problem of toxicity, concentrations can be increased as much as 300% to accommodate the amount of liquid in a particular food product. In addition, since the product is stable at a neutral pH, no flavors or fragrances are needed.

The composition comprises from about 0.05 to about 0.15 percent vegetable cellulose fiber of less than about 40 microns in size, from about 0.05 to about 0.1 percent beeswax, from about 0.0005 to about 0.002 percent soy lecithin and from about 1.0 to about 2.0 percent potassium sorbate, all by weight. The balance of the composition comprises water.

To manufacture the viscous liquid preservative, water is heated to between about 160 and 178 degrees F. Vegetable cellulose fiber is added to the heated water and agitated or mixed to a homogenous solution. Beeswax is then added and agitated or mixed to a homogenous solution. The soy lecithin is added and agitated or mixed to a homogenous solution. Finally, the potassium sorbate may be added to reach the desired pH. The composition is then filtered and cooled to at least 90 degrees F.

The invention accordingly comprises the features of construction, combination of elements, and arrangement of parts which will be exemplified in the construction hereinafter set forth, and the scope of the invention will be indicated in the claims.

### DETAILED DESCRIPTION OF THE PREFERRED EMBODIMENT

The present invention relates to a viscous liquid preservative formulated to reduce growth of microbial contaminants such as bacteria and spores that cause food spoilage.

The viscous liquid preservative comprises a composition of cellulose fiber, beeswax and an emulsifier having a neutral pH.

The cellulose fiber comprises a vegetable cellulose fiber; while, the emulsifier comprises soy lecithin.

This particular combination of food grade ingredients permits the composition to be altered or changed dependent of the food stuff or beverage to be preserved while adjusting the pH range to suit the food product or beverage being preserved. In particular, the cellulose fiber gives greater texture protection, the potassium sorbate adjusts the pH, the water adjusts the concentration, and the soy lecithin and beeswax controls the emulsification.

The composition comprises from about 0.05 to about 0.15 percent vegetable cellulose fiber, from about 0.05 to about 0.1 percent beeswax, from about 0.0005 to about 0.002 percent soy lecithin and from about 1.0 to about 2.0 percent potassium sorbate, all by weight. The balance of the composition comprises water.

The composition preferably comprises about 0.093 percent vegetable cellulose fiber of about 40 microns in size or less, about 0.067 percent beeswax and about 0.001 or less percent soy lecithin with the balance water, all by weight. The water preferably has a bacterial colony count of less than about 100 colonies per ML.

In addition, about 1.5 percent by weight potassium sorbate may be added to control the pH of the food or beverage to which the composition is added. The pH is at least about 7.0 and preferably from at least about 7.0 to about 7.8.

To manufacture the viscous liquid preservative, the water is heated to between about 160 and 178 degrees F. and preferably at least 165 degrees F. The vegetable cellulose fiber is added to the heated water and agitated or mixed to a homogenous solution that generally is accomplished in about 5 minutes. The beeswax is then added and agitated or mixed to a homogenous solution that generally takes about 2 minutes. The soy lecithin is added and agitated or mixed to a homogenous solution that generally takes about 5 minutes. Finally, the potassium sorbate may be added to reach the desired pH. The composition is then pumped through filters at a pressure no greater than about 15 psi and then cooled to at least about 90 degrees F.

It will thus be seen that the objects set forth above, among those made apparent from the preceding description are efficiently attained and since certain changes may be made in the above construction without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Now that the invention has been described,

What is claimed is:

1. A food and beverage preservative formulated to reduce growth of microbial contaminants such as bacteria and spores that cause food and beverage spoilage including a cellulose fiber and an emulsifier having a pH of at least about 7.0.

2. The food and beverage preservative of claim 1 wherein said cellulose fiber comprises vegetable cellulose fiber.

3. The food and beverage preservative of claim 2 wherein said emulsifier comprises soy lecithin.

4. The food and beverage preservative of claim 3 further comprising beeswax.

5. The food and beverage preservative of claim 2 further comprising beeswax.

6. The food and beverage preservative of claim 1 wherein said emulsifier comprises soy lecithin.

7. The food and beverage preservative of claim 1 further comprising beeswax.

8. The food and beverage preservative of claim 7 wherein said cellulose fiber comprises vegetable cellulose fiber.

9. A food and beverage preservative formulated to reduce growth of microbial contaminants such as bacteria and spores that cause food and beverage spoilage comprising from about 0.05 to about 0.15 percent vegetable cellulose fiber, from about 0.05 to about 0.1 percent beeswax, from about 0.0005 to about 0.002 percent soy lecithin with the balance water, all by weight.

10. The food and beverage preservative of claim 9 wherein said composition comprises about 0.093 percent vegetable cellulose fiber, about 0.067 percent beeswax and about 0.001 or less percentage soy lecithin with the balance water, all by weight.

11. The food and beverage preservative of claim 10 wherein the pH of said composition is at least about 7.0 to about 7.8.

12. The food and beverage preservative of claim 9 wherein the pH of said composition is at least about 7.0.

13. A food and beverage preservative formulated to reduce growth of microbial contaminants such as bacteria and spores that cause food and beverage spoilage comprising a vegetable cellulose fiber and an emulsifier.

14. The food and beverage preservative of claim 13 wherein said emulsifier comprises soy lecithin.

15. The food and beverage preservative of claim 14 further comprising beeswax.

16. The food and beverage preservative of claim 13 further comprising beeswax.

17. The food and beverage preservative of claim 13 wherein the pH of said composition is at least about 7.0.

18. The food and beverage preservative of claim 13, wherein the pH of said composition is at least about 7.0 to about 7.8.

19. The food and beverage preservative of claim 3 wherein the pH of said preservative is at least about 7.0 to about 7.8.

20. The food and beverage preservative of claim 4 wherein the pH of said preservative is at least about 7.0 to about 7.8.

21. The food and beverage preservative of claim 5 wherein the pH of said preservative is at least about 7.0 to about 7.8.

22. The food and beverage preservative of claim 1 wherein the pH of said preservative is at least about 7.0 to about 7.8.

23. A method of manufacturing a food and beverage preservative comprising the following steps: (1) heating water to between about 160 to about 178 degrees F.; (2) adding vegetable cellulose fiber and mixing to a substantially homogenous solution; (3) adding an emulsifier and mixing to a substantially homogenous solution and (4) adjusting the pH to at least about 7.0.

24. The method of manufacturing a food and beverage preservative of claim 23 further including the additional step of cooling said composition to at least about 90 degrees F.

25. The method of manufacturing a food and beverage preservative of claim 23 further including the additional step of filtering said composition through filters at a pressure no greater than about 15 psi

26. The method of manufacturing a food and beverage preservative of claim 25 further including the additional step of cooling said composition to at least about 90 degrees F.

27. The method of manufacturing a food and beverage preservative of claim 23 further comprising beeswax.

28. The method of manufacturing a food and beverage preservative of claim 27 wherein said emulsifier further comprises soy lecithin.

29. The method of manufacturing a food and beverage preservative of claim 23 wherein said emulsifier comprises soy lecithin.

30. The method of manufacturing a food and beverage preservative of claim 29 wherein said composition comprises from about 0.05 to about 0.15 percent vegetable cellulose fiber, from about 0.05 to about 0.1 percent beeswax and from about 0.0005 to about 0.002 percent soy lecithin with the balance water, all by weight.

31. The method of manufacturing a food and beverage preservative of claim 29 wherein said composition comprises about 0.093 percent vegetable cellulose fiber; about 0.067 percent beeswax and about 0.001 or less percent soy lecithin with the balance water, all by weight.

32. The method of manufacturing a food and beverage preservative of claim 31 wherein about 1.5 percent by weight potassium sorbate is added to control the pH.
